(19) United States
(12) Patent Application Publication
(10) Pub. No.: US 2023/0257484 A1
(43) Pub. Date: Aug. 17, 2023

(54) DENDROBIUM OFFICINALE OLIGOSACCHARIDE, DENDROBIUM OFFICINALE OLIGOSACCHARIDE DERIVATIVE AND PREPARATION METHOD AND USE THEREOF

(71) Applicants: KUNMING INSTITUTE OF BOTANY, CHINESE ACADEMY OF SCIENCES, Yunnan (CN); BEIJING DR PLANT BIOTECHNOLOGY CO., LTD., Beijing (CN)

(72) Inventors: Jiangmiao HU, Yunnan (CN); Liu YANG, Yunnan (CN); Yuhua WANG, Yunnan (CN); Rui HE, Yunnan (CN); Yong XIE, Yunnan (CN)

(21) Appl. No.: 17/788,422

(22) PCT Filed: Nov. 9, 2021
(86) PCT No.: PCT/CN2021/129570

§ 371 Date: Jun. 23, 2022

(30) Foreign Application Priority Data
Mar. 5, 2021 (CN) 202110247117.X

(51) Int. Cl. C08B 37/00; C07H 3/06; A61K 8/73; A61K 36/8984; A61K 31/716; A61Q 19/08; A61K 9/06; A61P 37/02; B01D 11/02; B01D 21/26

(52) U.S. Cl. CPC C08B 37/006; C07H 3/06; A61K 8/73; A61K 36/8984; A61K 31/716; A61Q 19/08; A61K 9/06; A61P 37/02; B01D 11/0288; B01D 21/262; A61K 2236/331; A61K 2236/333; A61K 2236/53; A61K 2236/51

(57) ABSTRACT

A Dendrobium officinale oligosaccharide, a Dendrobium officinale oligosaccharide derivative and a preparation method and use thereof. The Dendrobium officinale oligosaccharide includes 3-9 glycoside residues and a glucose residue at a non-reducing end; the derivative thereof is obtained by substituting with a hydrophobic residue at the non-reducing end; the preparation method and use of both are also provided.

### CROSS REFERENCE TO RELATED APPLICATION

This patent application is a national stage application of International Patent Application No. PCT/CN2021/129570, filed on Nov. 9, 2021 which claims the benefit and priority of Chinese Patent Application No. 202110247117.X filed on Mar. 5, 2021, the disclosure of which is incorporated by reference herein in its entirety as part of the present application.

### TECHNICAL FIELD

The present disclosure relates to the technical field of natural medicine chemistry, and in particular to a Dendrobium officinale oligosaccharide, a Dendrobium officinale oligosaccharide derivative and a preparation method and use thereof.

### BACKGROUND ART

Dendrobium officinale (Dendrobium officinale Kimura et Migo) is a perennial herb of the genus Dendrobium in the family Orchidaceae, which is listed in each edition of the Pharmacopoeia. According to the records in the Pharmacopoeia, it has the effects of such as engendering liquid and nourishing stomach, nourishing yin and clearing heat, moistening lung and boosting kidney, and brightening eyes and strengthening lumbus. Modern pharmacological research shows that Dendrobium officinale has antioxidative, hypoglycemic, immunomodulatory and anti-inflammatory activities. It is recorded in ancient literatures that the stem of Dendrobium officinale can be used for moisturizing skin and treating diabetes. Studies have shown that Dendrobium officinale mainly contains polysaccharides, stilbene, flavonoids, phenanthrene, lignin and other chemical components.

Aging is an inevitable result of human growth, and the way to delay aging has always been the ultimate proposition in the fields of skin care products and biomedicine. Aging is roughly divided into two categories: physiological aging and pathological aging. Physiological aging refers to the physiological degeneration process that occurs after the mature period, and pathological aging is the senility changes caused by various external factors (including various diseases). The two are actually difficult to distinguish. It is recorded in ancient literatures that Dendrobium officinale has the anti-aging effects, and the natural, green, safe and efficient anti-aging substances in Dendrobium officinale are thus the focus of research.

At present, many domestic and foreign researchers have conducted research on the extraction and separation of chemical components in various parts of Dendrobium officinale. Most of the researchers mainly obtain small-molecule phenol components or polysaccharides with a molecular weight of more than 100,000. Some researchers have isolated 20 compounds from Dendrobium officinale, mainly bibenzil components, and have found that the small-molecule phenol components of Dendrobium officinale have antioxidative, anti-inflammatory and anti-tumor effects through activity screening. Some researchers have extracted and separated two polysaccharides from Dendrobium officinale with a molecular weight of 7.4×10⁵ Da and 5.4×10⁵ Da, respectively. Some researchers have used a method of water extraction, alcohol precipitation and sulfuric acid hydrolysis to obtain polysaccharides from Dendrobium officinale, and have found that the polysaccharides of Dendrobium officinale influence the count of peripheral white blood cells in mice through activity screening, indicating an immunomodulatory effect. Some researchers have obtained polysaccharides from Dendrobium officinale by ultrasonic extraction and have found that the polysaccharides of Dendrobium officinale influence the SOD, MDA and CAT of Drosophila through activity screening, indicating an antioxidative activity. Some researchers have demonstrated that there are mainly four homogeneous polysaccharides among polysaccharides of Dendrobium officinale, with a molecular weight in a range of 6,800-1.78×10⁵ Da. The previous research work mostly concentrates on the study of the pharmacological activity of the small-molecule phenol components and the polysaccharides, whereas there are few reports on the research of oligosaccharides.

### SUMMARY

In view of this, the present disclosure provides a Dendrobium officinale oligosaccharide, a Dendrobium officinale oligosaccharide derivative and a preparation method and use thereof. It is illustrated that the Dendrobium officinale oligosaccharide has anti-aging and immunomodulatory effects, and is suitable for applying to skin cosmetics, health foods and medicines with anti-aging and immunomodulatory effects.

In order to achieve the above objectives, the present disclosure mainly provides the following technical solutions:

The present disclosure provides a Dendrobium officinale oligosaccharide, comprising 3-9 glycoside residues and a glucose residue at a non-reducing end.

In some embodiments, the Dendrobium officinale oligosaccharide comprises 6 glycoside residues.

The Dendrobium officinale oligosaccharide has a structure represented by formula (I):

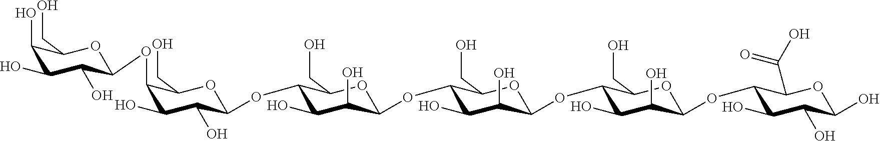

In some embodiments, the glycoside residue includes a galactose residue, a mannose residue and a glucose residue.

In some embodiments, a molar ratio of the galactose residue, the mannose residue and the glucose residue is (3-2):(2-1):(2-1).

In some embodiments, a molar ratio of the galactose residue, the mannose residue and the glucose residue is 2:1:1.

The present disclosure provides a Dendrobium officinale oligosaccharide derivative, where the Dendrobium officinale oligosaccharide derivative is generated by reacting the glucose residue of the Dendrobium officinale oligosaccharide according to the above technical solutions with a hydrophobic residue, and the hydrophobic residue includes any one of linear or branched C₁-C₁₈ alkyl, C₁-C₁₈ alkyl carboxylic acid, C₁-C₁₈ alkyl amine, C₁-C₁₈ aryl alkyl and C₁-C₁₈ alkyl amide, or

the hydrophobic residue includes any one of benzoyl, phenylacetyl and phenylpropionyl.

The present disclosure provides use of the Dendrobium officinale oligosaccharide or the Dendrobium officinale oligosaccharide derivative according to the above technical solutions in preparation of anti-aging or immunomodulatory products.

In some embodiments, the products include medicines, foods, formulations and cosmetics.

In some embodiments, a type of the cosmetics includes a face cream, an emulsion, a toning lotion or a repair cream.

The present disclosure provides a method for extracting the Dendrobium officinale oligosaccharide described in above technical solutions, comprising:

1) preparation of a Dendrobium officinale powder: drying a stem of Dendrobium officinale, then smashing and sieving to obtain a Dendrobium officinale powder;

2) preparation of a water extract of Dendrobium officinale: mixing the Dendrobium officinale powder with water, then subjecting to a boiling water bath for one or more times, and then merging water extracts to obtain a water extract of Dendrobium officinale;

3) preparation of a concentrated water extract of Dendrobium officinale: centrifuging the water extract of Dendrobium officinale, and then concentrating under reduced pressure to obtain a concentrated water extract of Dendrobium officinale;

4) preparation of a redissolving liquid: adding ethanol to the concentrated water extract of Dendrobium officinale, precipitating, centrifuging and then discarding a supernatant to obtain a precipitate; adding water to the obtained precipitate for redissolving to obtain a redissolving liquid;

5) preparation of an aqueous solution of Dendrobium officinale oligosaccharide: deproteinizing the redissolving liquid, and freeze-drying to obtain a crude polysaccharide of Dendrobium officinale; adding water to dissolve the crude polysaccharide of Dendrobium officinale, then adding cellulase for a reaction, followed by inactivating and centrifuging, and discarding a precipitate to obtain an aqueous solution of Dendrobium officinale oligosaccharide;

6) preparation of the Dendrobium officinale oligosaccharide: purifying the aqueous solution of Dendrobium officinale oligosaccharide to obtain the Dendrobium officinale oligosaccharide.

In some embodiments, in step 1), a temperature for the drying is 50-60° C.; a moisture mass content of the stem of Dendrobium officinale after the drying is less than 6%; a mesh number of the sieving is 20-40 meshes;

in step 2), the Dendrobium officinale powder and water are mixed in a mass-volume ratio of 1:(50-80) g/mL; the boiling water bath is performed for 2-3 times; a time for each boiling water bath is 1-2 h;

in step 3), the centrifugation is conducted under conditions of 3000-4000 rpm/min, 8-12 min and room temperature; the concentration under reduced pressure is conducted under conditions of a rotary evaporation method, an ethanol recycling temperature of less than 50° C., and a pressure of 0.7 Pa-0.9 Pa; a material-liquid mass-volume ratio of the concentrated water extract of Dendrobium officinale is 1:(5-10) g/mL.

In some embodiments, in step 4), the addition of ethanol to the concentrated water extract of Dendrobium officinale and the precipitation are specifically conducted as follows: adding ethanol with a volume concentration of 95% to the concentrated water extract of Dendrobium officinale until an alcohol concentration of a mixture is 70%-80% in volume percentage, and then precipitating overnight;

the centrifugation is conducted under conditions of 3500-4500 rpm, 18-22 min and room temperature; a weight-volume ratio of the precipitate and water for redissolving is 1:(4-6) g/mL;

in step 5), the deproteinization of the redissolving liquid is conducted by a Sevage method;

a temperature for the freeze-drying is 45° C.;

the addition of water to dissolve the crude polysaccharide of Dendrobium officinale is conducted as follows: adding water to the crude polysaccharide of Dendrobium officinale for dissolving until 4.5-5.5 mg/mL;

an added amount of the cellulase is 2%-4% of a weight of the crude polysaccharide of Dendrobium officinale;

after adding the cellulase, the reaction is conducted under a reaction temperature of 55-65° C. for a reaction time of 2 h-4 h;

the inactivation is conducted by boiling for 25-35 min;

the centrifugation is conducted under conditions of 3500-4500 rpm, 15-25 min and room temperature;

an oligosaccharide content in the aqueous solution of Dendrobium officinale oligosaccharide is greater than 80%;

in step 6), the purification is conducted by passing through a G75 gel column; a purity of the Dendrobium officinale oligosaccharide is 99.86%.

The present disclosure provides a method for preparing the Dendrobium officinale oligosaccharide derivative described in the above technical solutions, comprising:

1) reducing a fatty acid into a fatty alcohol;

2) glycosylating the fatty alcohol with the Dendrobium officinale oligosaccharide to obtain the Dendrobium officinale oligosaccharide derivative.

In some embodiments, in step 1), a reaction process of the reduction of a fatty acid into a fatty alcohol is as follows: mixing 9-11 mmol of the fatty acid, 18-22 mmol of NaBH₄, 38-42 mmol of AlCl₃, 25-29 ml of THF, and 2-4 ml of MeOH for a reaction, and the reaction is conducted under a temperature of 95-105° C. for a time of 7.5-8.5 h with a yield of 88-92%;

in step 2), a reaction process of the glycosylation is as follows: mixing the alcohol and the saccharide in a molar ratio of 1:(8-10), and then using cetyl ammonium bromide as a catalyst for catalyzation, and the reaction is conducted under a temperature of 110-115° C. for a time of 2.5-3.5 h with a yield of 78-82%.

The present disclosure provides an anti-aging or immunomodulatory product, where the anti-aging or immunomodulatory product is a face cream, an emulsion, a toning lotion or a repair cream;

the face cream includes in mass percentage: 0.4-0.6% of a Dendrobium officinale stock solution rich in oligosaccharides, 7.5-8.5% of stearic acid, 1.5-2.5% of C₁₆ alcohol, 1.5-2.5% of self-emulsifying monoglyceride, 1.5-2.5% of hydrogenated lanolin, 11-13% of liquid paraffin, 6-8% of glycerin, 1-2% of an emulsifier, 0.1-0.3% of a preservative, 0.1-0.3% of an essence, and the balance of water;

the emulsion includes in mass percentage: 0.4-0.6% of a Dendrobium officinale stock solution rich in oligosaccharides, 1.3-1.5% of stearic acid, 0.08-0.12% of cetyl alcohol, 1.7-1.9% of 2-ethyl alcohol cetyl stearate, 0.1-0.3% of isopropyl myristate, 0.9-1.1% of 2-hexyl-1-decanol, 7-8% of liquid paraffin, 2.5-3.5% of glycerol, 7-9% of propylene glycol, 0.08-0.12% of triethanolamine, 0.3-0.4% of a carboxyvinyl polymer, 1.5-2.5% of Arlacel 165, 0.1-0.3% of a preservative, 0.1-0.3% of an essence, and the balance of water;

the toning lotion includes in mass percentage: 1-10% of a Dendrobium officinale stock solution rich in oligosaccharides, 0.3-0.4% of sodium polyacrylate, 3.5-4.5% of glycerol, 2-3% of 1,3-butanediol, 0.4-0.6% of vitamin B₅, 0.4-0.6% of arbutin, 0.04-0.06% of EDTA-Na₂, and the balance of deionized water;

the repair cream includes in mass percentage: 5-7% of glycerin, 1.4-1.6% of carbomer, 1.4-1.6% of triethanolamine, 5.5-6.5% of propylene glycol, 0.15-0.25% of ethyl p-hydrobenzoate, 9-11% of a Dendrobium officinale stock solution rich in oligosaccharides, 0.4-0.6% of an essential oil, and the balance of deionized water.

Compared with the prior art, the Dendrobium officinale oligosaccharide, the Dendrobium officinale oligosaccharide derivative and the preparation method and use thereof in the present disclosure have at least the following beneficial effects:

In one aspect, the present disclosure illustrates for the first time the Dendrobium officinale oligosaccharides with a purity of more than or equal to 99% prepared from Dendrobium officinale, and carries out structural analysis to identify the structure of Dendrobium officinale oligosaccharides.

Further, the present disclosure conducts for the first time a systematic study on the anti-aging activity of Dendrobium officinale oligosaccharides, and has found that Dendrobium officinale oligosaccharides can well inhibit collagenase and the like and thus have a strong anti-aging effect. Moreover, they also have a certain activity on TNF-α and IL-6, and a good immunomodulatory effect.

In another aspect, the preparation method and active application of Dendrobium officinale oligosaccharides involved in the present disclosure provide a scientific basis for the application of Dendrobium officinale in related directions.

In yet another aspect, the Dendrobium officinale oligosaccharide prepared by the present disclosure can be used as an intermediate for the development of Dendrobium officinale series products (such as application in the fields of medicine, food, daily necessities, etc.).

### BRIEF DESCRIPTION OF THE DRAWINGS

FIGS. 1-3 show the structure and purity identification HPLC-ELSD, NMR and MS spectra of Dendrobium officinale oligosaccharides of the present disclosure:

FIG. 1 shows the purity identification HPLC-ELSD spectrum of Dendrobium officinale oligosaccharides;

FIG. 2 shows the ¹H-NMR and ¹³C-NMR spectra of Dendrobium officinale oligosaccharides, where FIG. 2A is a ¹H-NMR spectrum, and FIG. 2B is a ¹³C-NMR spectrum;

FIGS. 3-6 show the test results of the anti-aging activity of Dendrobium officinale oligosaccharides:

FIG. 3 shows the anti-oxidation rate of Dendrobium officinale oligosaccharides based on the scavenging rate of DPPH free radicals (oligosaccharide concentrations of 4%, 8%, 11%, 15%, 20%); where the ordinate is the anti-oxidation rate % represented by the scavenging rate of DPPH free radicals, and the abscissa (from 1 to 5) corresponds to the oligosaccharide concentration of 4%, 8%, 11%, 15%, 20%, respectively;

FIG. 4 shows the inhibition rate of Dendrobium officinale oligosaccharides on elastase activity (oligosaccharide concentrations of 4%, 8%, 11%, 15%, 20%); where the ordinate is the elastase inhibition rate %, and the abscissa (from 1 to 5) corresponds to the oligosaccharide concentration of 4%, 8%, 11%, 15%, 20%, respectively;

FIG. 5 shows the inhibition rate of Dendrobium officinale oligosaccharides on collagenase activity (oligosaccharide concentrations of 0.4%, 0.8%, 1%, 2%, 4%); where the ordinate is the collagenase inhibition rate %, and the abscissa (from 1 to 5) corresponds to the oligosaccharide concentration of 0.4%, 0.8%, 1%, 2%, 4%, respectively;

FIG. 6 shows the activity of Dendrobium officinale oligosaccharides on promoting the secretion of hyaluronic acid (oligosaccharide concentrations of 4%, 8%, 11%, 15%, 20%); where the ordinate is the secretion of hyaluronic acid (ng/mL), and the abscissa (from 1 to 5) corresponds to the oligosaccharide concentration of 4%, 8%, 11%, 15%, 20%, respectively;

FIG. 7 shows the effect of Dendrobium officinale oligosaccharides on the immunomodulatory activity of RAW264.7 cells (oligosaccharide concentrations of 3.125 μg/mL, 6.25 μg/mL, 12.5 μg/mL, 50 μg/mL, 100 μg/mL, 200 μg/mL); where A in FIG. 7 shows the effect of oligosaccharides on TNF-α secretion, the ordinate is TNF-α secretion pg/mL and the abscissa (from 1 to 5) corresponds to blank, lipopolysaccharide LPS and oligosaccharide with a final reaction concentration of 12.5 μg/mL, 6.25 μg/mL, 3.125 μg/mL, respectively; B in FIG. 7 shows the effect of oligosaccharides on IL-6 secretion, the ordinate is IL-6 secretion pg/mL, and the abscissa (from 1 to 5) corresponds to blank, lipopolysaccharide LPS and oligosaccharide with a final reaction concentration of 200 μg/mL, 100 μg/mL, 50 μg/mL, respectively.

### DETAILED DESCRIPTION OF THE EMBODIMENTS

In order to further explain the technical means and effects of the present disclosure to achieve the intended purpose of the present disclosure, the specific implementations, structures, features and effects of the disclosure according to the present disclosure will be described in detail below with reference to the accompanying drawings and preferred embodiments. In the following description, different “one embodiment” or “an embodiment” do not necessarily refer to the same embodiment. In addition, specific features, structures or characteristics in one or more embodiments may be combined in any suitable form.

The term “skin care product” in the present disclosure may refer to a skin care toner, a skin care lotion, a skin care mask, a skin cream, etc.; the skin care products in the embodiments of the present disclosure can also refer to cosmetics.

The present disclosure provides a Dendrobium officinale oligosaccharide, comprising 3-9 glycoside residues and a glucose residue at a non-reducing end; a Dendrobium officinale oligosaccharide containing 6 glycoside residues has a structure represented by formula (I):

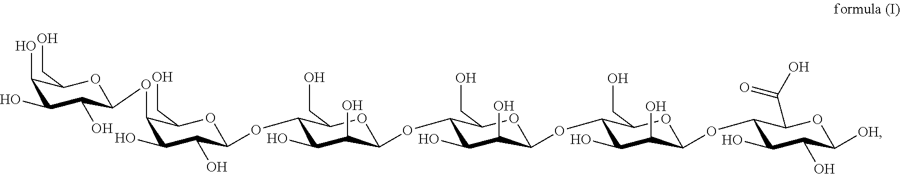

Herein, the glycoside residue includes a galactose residue, a mannose residue and a glucose residue; a molar ratio of the galactose residue, the mannose residue and the glucose residue is (3-2):(2-1):(2-1), preferably 2:1:1.

The present disclosure provides a method for extracting the Dendrobium officinale oligosaccharide described in above technical solutions, comprising:

1) preparation of Dendrobium officinale powder: drying a stem of Dendrobium officinale, then smashing and sieving to obtain the Dendrobium officinale powder;

In some embodiments, a temperature for the drying is 50-60° C.; a moisture mass content of the stem of Dendrobium officinale after the drying is less than 6%; and a mesh number of the sieving is 20-40 meshes;

2) preparation of a water extract of Dendrobium officinale: mixing the Dendrobium officinale powder with water, then subjecting to a boiling water bath for one or more times, and merging the water extracts obtained after the boiling water bath to obtain a water extract of Dendrobium officinale;

in some embodiments, the Dendrobium officinale powder and water are mixed according to a mass-volume ratio of 1:(50-80) g/mL; the boiling water bath is performed for 2-3 times; and a time for each boiling water bath is 1-2 h;

3) preparation of a concentrated water extract of Dendrobium officinale: centrifuging the water extract of Dendrobium officinale, and then concentrating under reduced pressure to obtain a concentrated water extract of Dendrobium officinale;

in some embodiments, the centrifugation is conducted under 3000-4000 rpm/min, 8-12 min, and room temperature; the concentration under reduced pressure is conducted by using a rotary evaporation method, an ethanol recycling temperature of less than 50° C. and a pressure of 0.7 Pa-0.9 Pa; a material-liquid mass-volume ratio of the concentrated water extract of Dendrobium officinale is 1:(5-10) g/mL;

4) preparation of a redissolving liquid: adding ethanol to the concentrated water extract of Dendrobium officinale, precipitating, centrifuging and then discarding a supernatant to obtain a precipitate; and then adding water to the obtained precipitate for redissolving to obtain a redissolving liquid;

in some embodiments, ethanol with a volume concentration of 95% is added to the concentrated water extract of Dendrobium officinale until an alcohol concentration of the mixture is 70%-80% by volume percentage, and then the overnight precipitation is carried out; the conditions of centrifugation are 3500-4500 rpm, 18-22 min, and room temperature; and water is added according to a weight-volume ratio of the precipitate and water of 1:(4-6) g/mL for redissolving;

5) preparation of an aqueous solution of Dendrobium officinale oligosaccharide: deproteinizing the redissolving liquid, and freeze-drying to obtain a crude polysaccharide of Dendrobium officinale; and adding water to dissolve the crude polysaccharide of Dendrobium officinale, then adding cellulase for a reaction, followed by inactivating and centrifuging, and discarding a precipitate to obtain an aqueous solution of Dendrobium officinale oligosaccharide;

in some embodiments, the redissolving liquid is deproteinized by a Sevage method; the freeze-drying temperature is 45° C.; adding water to dissolve the crude polysaccharide of Dendrobium officinale to 4.5-5.5 mg/mL; an added amount of the cellulase is 2%-4% of the weight of the crude polysaccharide of Dendrobium officinale; after the cellulase is added, the reaction is conducted under a reaction temperature of 55-65° C., and a reaction time of 2 h-4 h; inactivating by boiling for 25-35 min; the centrifugation are conducted under 3500-4500 rpm, 15-25 min, and room temperature; the oligosaccharide content in the aqueous solution of Dendrobium officinale oligosaccharide is greater than 80%;

6) preparation of Dendrobium officinale oligosaccharide: purifying the aqueous solution of Dendrobium officinale oligosaccharide to obtain the Dendrobium officinale oligosaccharide;

in some embodiments, the purification is conducted by using a G75 gel column; the purity of the Dendrobium officinale oligosaccharide is 99.86%.

The present disclosure provides use of a Dendrobium officinale oligosaccharide described in above technical solutions in the preparation of anti-aging or immunomodulatory products.

In some embodiments, the products include medicines, foods, formulations and cosmetics. In some embodiments, the cosmetics include a face cream, an emulsion, a toning lotion or a repair cream.

The present disclosure provides a Dendrobium officinale oligosaccharide derivative, where the Dendrobium officinale oligosaccharide derivative is generated by reacting the glucose residue of the Dendrobium officinale oligosaccharide according to the above technical solutions with a hydrophobic residue, and the hydrophobic residue includes any one of linear or branched C₁-C₁₈ alkyl, C₁-C₁₈ alkyl carboxylic acid, C₁-C₁₈ alkyl amine, C₁-C₁₈ aryl alkyl and C₁-C₁₈ alkyl amide, or

the hydrophobic residue includes any one of benzoyl, phenylacetyl, and phenylpropionyl (Replacing hydroxy and methoxy with phenyl).

The present disclosure provides a method for extracting the Dendrobium officinale oligosaccharide derivative described in the above technical solutions, comprising:

1) reducing a fatty acid into a fatty alcohol;

in some embodiments, a reaction process of reducing the fatty acid into the fatty alcohol is as follows: mixing 10 mmol of fatty acid, 20 mmol of NaBH₄, 40 mmol of AlCl₃, 27 ml of THF, 3 ml of MeOH for a reaction, the reaction temperature is 100° C., the reaction time is 8 h, and the yield is 90%;

2) glycosylating the fatty alcohol with Dendrobium officinale oligosaccharide;

in some embodiments, performing the glycosylation reaction of fatty alcohols and Dendrobium officinale oligosaccharides obtained by reduction, the glycosylation reaction is conducted as follows: a molar ratio of alcohol to saccharide is 1:(8-10), the catalyst is cetylammonium bromide, the reaction temperature is 110-115° C., the reaction time is 3 h, and the yield is 80%.

The present disclosure provides use of a Dendrobium officinale oligosaccharide derivative described in above technical solutions in the preparation of anti-aging or immunomodulatory products.

In some embodiments, the products include medicines, foods, formulations and cosmetics; in some embodiments, the cosmetics include a face cream, an emulsion, a toning lotion or a repair cream.

The present disclosure provides a method for separating and purifying oligosaccharides from Dendrobium officinale. The polysaccharide of Dendrobium officinale has a large molecular weight (more than or equal to 100,000) and is difficult to be absorbed through skin cells, and thus mainly acts on the outside of the skin and has a moisturizing effect, while the oligosaccharide formed by enzymatic hydrolysis of polysaccharides has a small molecular weight and is easily absorbed through skin cells, which plays an anti-aging effect. According to the different molecular weights of the two, they can be used differently.

On the other hand, it is also found that Dendrobium officinale oligosaccharides can increase the survival rate and survival time of Hacat and RAW264.7 cells. Considering that Dendrobium officinale oligosaccharides may have good anti-aging activities and immunomodulatory activities, the activities on several main target proteins (elastase, collagenase, hyaluronic acid) contributing to aging have been verified. The results show that Dendrobium officinale oligosaccharides have a strong inhibitory effect on collagenase and can effectively alleviate the degradation and metabolism of collagen, thus maintaining the youth state of skin. They also have a certain inhibitory effect on elastase, and meanwhile a certain activity to promote the secretion of hyaluronic acid. Dendrobium officinale oligosaccharides also have a certain activity on TNF-α and IL-6, and have a good immunomodulatory effect.

The present disclosure provides an anti-aging or immunomodulatory product, and the anti-aging or immunomodulatory product is a face cream or an emulsion.

When the anti-aging or immunomodulatory product is a face cream, it includes components in mass percentage: 0.4-0.6% of Dendrobium officinale stock solution rich in oligosaccharides, 7.5-8.5% of stearic acid, 1.5-2.5% of C₁₆ alcohol, 1.5-2.5% of self-emulsifying monoglyceride, 1.5-2.5% of hydrogenated lanolin, 11-13% of liquid paraffin, 6-8% of glycerin, 1-2% of an emulsifier, 0.1-0.3% of a preservative, 0.1-0.3% of an essence, and the balance of water.

When the anti-aging or immunomodulatory product is an emulsion, it includes components in mass percentage: 0.4-0.6% of Dendrobium officinale stock solution rich in oligosaccharides, 1.3-1.5% of stearic acid, 0.08-0.12% of cetyl alcohol, 1.7-1.9% of 2-ethyl alcohol cetyl stearate, 0.1-0.3% of isopropyl myristate, 0.9-1.1% of 2-hexyl-1-decanol, 7-8% of liquid paraffin, 2.5-3.5% of glycerol, 7-9% of propylene glycol, 0.08-0.12% of triethanolamine, 0.3-0.4% of a carboxyvinyl polymer, 1.5-2.5% of Arlacel 165, 0.1-0.3% of a preservative, 0.1-0.3% of an essence, and the balance of water.

The present disclosure provides an anti-aging or immunomodulatory product, and the anti-aging or immunomodulatory product is toner. In the present disclosure, the toning lotion includes components in mass percentage: 1-10% of Dendrobium officinale stock solution rich in oligosaccharides, 0.3-0.4% of sodium polyacrylate, 3.5-4.5% of glycerol, 2-3% of 1,3-butanediol, 0.4-0.6% of vitamin B₅, 0.4-0.6% of arbutin, 0.04-0.06% of EDTA-Na₂, and the balance of deionized water.

In the present disclosure, a method for preparing the toning lotion is as follows: dissolving sodium polyacrylate in water and stirring for fully swelling, and then adding the remaining components and continuing stirring; subsequently, adding the Dendrobium officinale stock solution rich in oligosaccharides and stirring, and then adding deionized water to a constant volume to obtain the toning lotion.

The present disclosure provides an anti-aging or immunomodulatory product, and the anti-aging or immunomodulatory product is a repair cream; in the present disclosure, the repair cream includes components in mass percentage: 5-7% of glycerin, 1.4-1.6% of carbomer, 1.4-1.6% of triethanolamine, 5.5-6.5% of propylene glycol, 0.15-0.25% of ethyl p-hydrobenzoate, 9-11% of Dendrobium officinale stock solution rich in oligosaccharides, 0.4-0.6% of an essential oil, and the balance of deionized water.

In the present disclosure, a method for preparing the repair cream is as follows: adding carbomer into an appropriate amount of deionized water, stirring uniformly, and standing overnight for fully swelling; then adding glycerin, and adjusting the pH value with triethanolamine to increase viscosity of the gel matrix to obtain a carbomer gel;

mixing the Dendrobium officinale stock solution rich in oligosaccharides, propylene glycol and deionized water, then adding the carbomer gel, essential oil and ethyl p-hydrobenzoate thereto and stirring to an uniform and delicate state; adding distilled water to a sufficient amount, and grinding evenly to obtain the repair cream.

Hereinafter, the present disclosure is further described in detail with reference to the specific examples as follows.

### Example 1

In this example, substantially, Dendrobium officinale oligosaccharides were separated and purified from a stem of Dendrobium officinale, and further subjected to structural identification:

The fresh stem of Dendrobium officinale was cut and placed in a blast drying box to dry at 55° C., and the moisture content was measured. When the moisture content reached 6% or less, the stem was smashed and passed through a 20-40 mesh sieve; 50 g of a Dendrobium officinale powder was taken, 2500 mL of ultrapure water was added thereto, then the mixture was subjected to boiling water bath for 2 times with 2 h each time, and the water extracts were merged. Then a centrifugation was conducted at 3000-4000 rpm for 10 min at room temperature of 25° C. to obtain a water extract of Dendrobium officinale; the water extract of Dendrobium officinale was concentrated under reduced pressure to 500 mL to obtain a concentrated water extract of Dendrobium officinale. An appropriate amount of 95% ethanol was added to the concentrated water extract of Dendrobium officinale until an alcohol concentration of the mixture was 70-80%, then the mixture was precipitated overnight, centrifuged at 4000 rpm for 20 min at room temperature of 25° C., and the supernatant was discarded to obtain a precipitate; water was added according to the ratio of the weight of the precipitate to the volume of water of 1:5 (m:v) (g/mL) for redissolving to obtain a redissolving liquid. The redissolving liquid was deproteinized by the Sevage method, and then freeze-dried to obtain a crude polysaccharide of Dendrobium officinale. 200 mg of the crude polysaccharide of Dendrobium officinale was taken and dissolved with water to 5 mg/mL, cellulase was added thereto in 2%-4% of the weight of the polysaccharide of Dendrobium officinale and mixed evenly, then the mixture was reacted at 60° C. for 2 h-4 h, boiled for 30 min for inactivation, centrifuged at 4000 rpm for 20 min at room temperature of 25° C., and a precipitate was discarded to obtain an aqueous solution of Dendrobium officinale oligosaccharide (the content of oligosaccharides in the aqueous solution of Dendrobium officinale oligosaccharide is greater than 80%). An appropriate amount of the aqueous solution of Dendrobium officinale oligosaccharide was taken and passed through a G75 gel column to obtain 150 mg of purified Dendrobium officinale oligosaccharide (with a purity of 99.86% and a molecular weight of about 1,000).

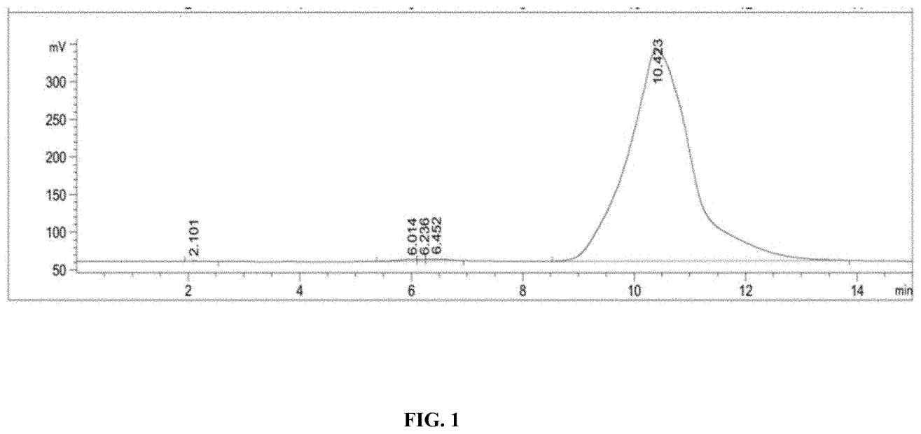

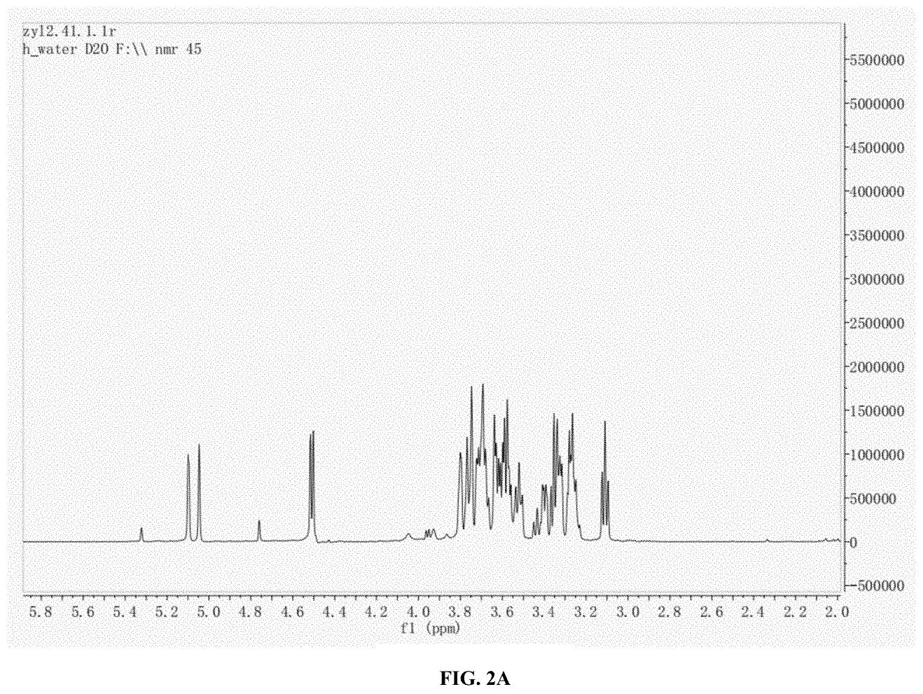

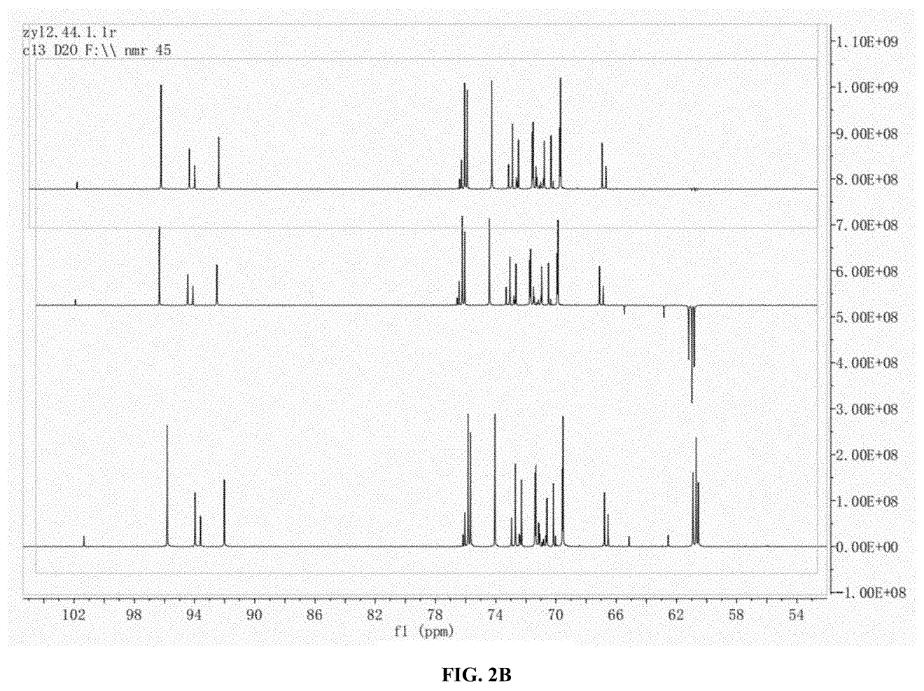

The purity of the Dendrobium officinale oligosaccharides was identified by high performance liquid chromatography, and the results are shown in FIG. 1. The structure of Dendrobium officinale oligosaccharides was identified by hydrogen nuclear magnetic resonance analysis and C13 nuclear magnetic resonance analysis, and the results are shown in FIG. 2.

### Example 2

Experiment on the DPPH Free Radical Scavenging

1. Reagents:

DPPH, water-soluble vitamin E (Trolox) (Trolox dissolved in absolute ethanol, with a concentration of 10 mM, stored at −20° C.), pure water.

2. Experimental Method:

The drug to be tested was mixed with DPPH (a final concentration of 100 M) and reacted at 30° C. for 1 h. 3 repeating wells were set, and a blank control well without drug and a Trolox positive control well were set at the same time. The OD value was measured by a microplate reader with a detection wavelength of 515 nm, and the anti-oxidation rate was calculated.

Anti-oxidation rate (%)=(1−experimental well OD₅₁₅ₙₘ/blank well OD₅₁₅ₙₘ)×100%.

3. Experimental Results:

As shown in Table 1.

TABLE 1

| | Final reaction | Anti-oxidation |
|---|---|---|
| Sample | concentration | rate (%) |
| Trolox | 25 | 95.668% |
| Dendrobium officinale | 4% | 11.335% |
| oligosaccharide | | |
| Dendrobium officinale | 8% | 14.346% |
| oligosaccharide | | |
| Dendrobium officinale | 11% | 16.213% |
| oligosaccharide | | |
| Dendrobium officinale | 15% | 21.155% |
| oligosaccharide | | |
| Dendrobium officinale | 20% | 25.217% |
| oligosaccharide | | |

4. Conclusion:

According to the data in Table 1, FIG. 3 is made for a clearer comparison. As can be seen from FIG. 3 and Table 1, the scavenging ability of DPPH free radicals of Dendrobium officinale oligosaccharides is evaluated in the experiment. The results show that the Dendrobium officinale oligosaccharides have a certain anti-oxidation rate (>20%) at a concentration of 15%-20%.

### Example 3

Experiment on the Elastase Inhibition Rate

1. Reagents:

Sample, positive control of catechin, Tris, HCl, porcine pancreatic elastase, N-succinyl-alanine-alanine-alanine-p-nitroaniline, DMSO.

2. Experimental Method:

(1) Preparation of Solution

Preparation of a sample solution: 1 mg/mL sample solution was prepared (pure water or a buffer solution was used for preparation, and if insoluble in water or the buffer solution, the sample was firstly dissolved with DMSO, and then water or the buffer solution was added. In principle, the content of DMSO should not exceed 5%).

Preparation of a positive control: 1 mg/mL catechin;

Preparation of a buffer solution: 0.1 M of Tris-HCl buffer solution, PH=8.0;

Preparation of a reaction substrate solution: N-succinyl-alanine-alanine-alanine-p-nitroaniline (AAAPVN) solution (2 mM);

Preparation: 28.5 μL of porcine pancreatic elastase solution (0.171 U/mL) was taken and dissolved in 10 mL of Tris-HCl buffer solution.

(2) The solution of 50 μL of sample, 50 μL of porcine pancreatic elastase and 100 μL of reaction substrate was added to a 96-well plate.

(3) After keeping at room temperature for 5 min, an absorbance at 420 nm was measured.

Elastase inhibition rate (%)=(1−sample OD_{420 nm}/experimental well OD₄₂₀ nm)×100%.

3. Experimental Results:

As shown in Table 2.

TABLE 2

| | Final reaction | Elastase inhibition |
|---|---|---|
| Sample | concentration | rate % |
| epicatechin gallate | 100 μg/ml | 68.540% |
| Dendrobium officinale | 4% | −2.135% |
| oligosaccharide | | |
| Dendrobium officinale | 8% | 3.886% |
| oligosaccharide | | |
| Dendrobium officinale | 11% | 8.417% |
| oligosaccharide | | |
| Dendrobium officinale | 15% | 9.723% |
| oligosaccharide | | |
| Dendrobium officinale | 20% | 15.827% |
| oligosaccharide | | |

4. Conclusion:

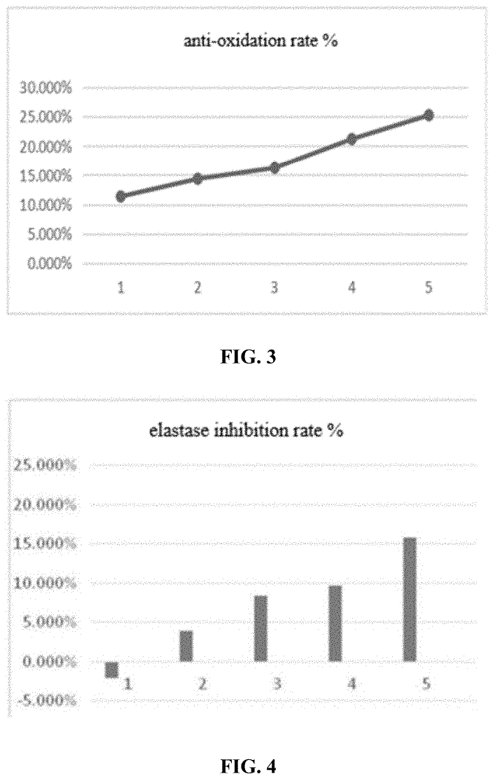

According to the data in Table 2, FIG. 4 is made for a clearer comparison. As can be seen from FIG. 4 and Table 2, the elastase inhibition rate of Dendrobium officinale oligosaccharides is evaluated in the experiment. The results show that the Dendrobium officinale oligosaccharides have a certain elastase inhibition activity (>10%) at a concentration of 20%.

### Example 4

Experiment on Collagenase Inhibition Rate

1. Reagents:

Protease buffer solution, protease, protease substrate, inhibitor (1,10-Phenanthroline). All reagents were stored at −20° C.

2. Experimental Method:

(1) Solution Preparation:

The collagenase reaction mixture is shown in Table 3.

TABLE 3

| Composition | Content (%) |
|---|---|
| Collagenase buffer solution | 60 |
| Collagenase substrate | 40 |

(2) The Compositions were Designed as Follows:

a. Background blank well: 100 μL/well of protease buffer solution.

b. Sample well: 100 μL, prepared into different concentrations.

c. Positive control well: 10 μL of protease+90 μL of protease buffer solution.

d. Inhibitor control well: 10 μL of protease+2 μL of inhibitor+88 μL of protease buffer solution as shown in Table 4.

TABLE 4

| | | Positive | Inhibitor | |
|---|---|---|---|---|
| | Sample | control | control | Background |
| | well | well | well | blank well |
| Group | (μl) | (μl) | (μl) | (μl) |
| Sample to be tested | 1-90 | — | — | — |
| Protease | 10 | 10 | 10 | — |
| Inhibitor | — | — | 2 | — |
| Protease buffer | Diluted | 90 | 88 | 100 |
| solution | to 100 | | | |

(3) After adding reagents according to the above groups, 100 μL of collagenase reaction mixture was added to each well, incubated in the dark, and then the OD value at 345 nm was measured.

The incubation time depended on the activity of the sample to be tested, and the low activity sample could be incubated for 1-3 h.

Collagenase inhibition rate (%)=(OD_{background well}−OD_{sample well})/(OD_{background well}−OD_{positive control well}).

3. Experimental Results:

As shown in Table 5.

TABLE 5

| | | Collagenase |
|---|---|---|
| | Final reaction | inhibition rate |
| Sample | concentration | (%) |
| Positive inhibitor | 1% | 102.23% |
| Dendrobium officinale | 0.4% | 50.32% |
| oligosaccharide | | |
| Dendrobium officinale | 0.8% | 63.63% |
| oligosaccharide | | |
| Dendrobium officinale | 1% | 72.20% |
| oligosaccharide | | |
| Dendrobium officinale | 2% | 84.80% |
| oligosaccharide | | |
| Dendrobium officinale | 4% | 110.54% |
| oligosaccharide | | |

4. Conclusion:

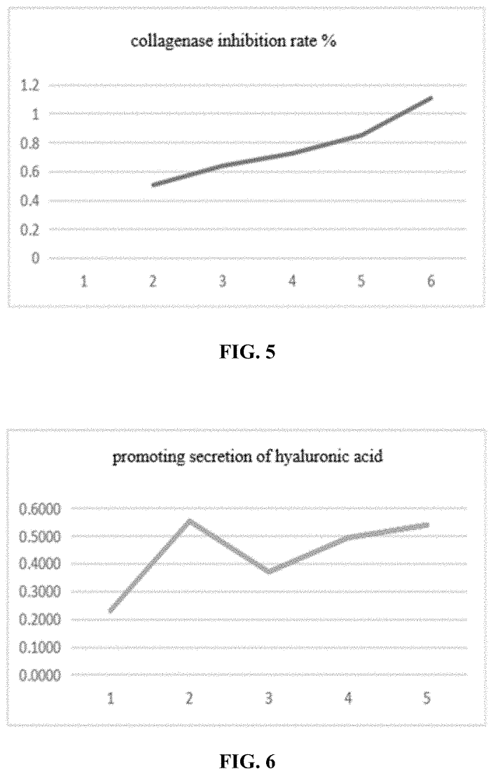

According to the data in Table 5, FIG. 5 is made for a clearer comparison. As can be seen from FIG. 5 and Table 5, the collagenase inhibition rate of Dendrobium officinale oligosaccharides is evaluated in the experiment. The results show that the Dendrobium officinale oligosaccharides have a strong collagenase inhibition activity (>50%) at a concentration of 0.4%-4%.

### Example 5

Experiment on Promoting Secretion of Hyaluronic Acid

1. Reagents and Cells:

Human immortalized cortical cells (Hacat cells), human hyaluronic acid Elisa kit, distilled water.

2. Experimental Method:

A 96-well plate was inoculated with 100 mL of cell suspension per well, and a concentration of the suspension was 4×10⁵ cells/mL. After 24 h of incubation, the original culture medium was discarded, then the cells were washed with PBS for 1-2 times and the serum-free medium containing different concentrations of the drug to be tested was added thereto, and continued to incubate for 24 h. A blank control without drugs and a TGF-β positive control were set. Then the cells were lysed with ripa lysis buffer solution containing 1% PMSF, the supernatant after centrifugation was taken, and the secretion of hyaluronic acid was detected according to the method provided in the collagen ELISA kit. The OD value was measured by a microplate reader with a detection wavelength of 450 nm. The OD value was substituted into the regression equation of the standard curve to calculate the increase rate of the secretion of hyaluronic acid.

3. Experimental Results:

As shown in Table 6.

TABLE 6

| | Final reaction | Secretion of hyaluronic |
|---|---|---|
| Sample | concentration | acid (ng/mL) |
| Blank control | | 0.2508 |
| Dendrobium officinale | 4% | 0.2294 |
| oligosaccharide | | |
| Dendrobium officinale | 8% | 0.5530 |
| oligosaccharide | | |
| Dendrobium officinale | 11% | 0.3691 |
| oligosaccharide | | |
| Dendrobium officinale | 15% | 0.4930 |
| oligosaccharide | | |
| Dendrobium officinale | 20% | 0.5392 |
| oligosaccharide | | |

4. Conclusion:

According to the data in Table 6, FIG. 6 is made for a clearer comparison. As can be seen from FIG. 6 and Table 6, the secretion-promoting ability of hyaluronic acid of Dendrobium officinale oligosaccharides is evaluated. The results show that the Dendrobium officinale oligosaccharides have a certain activity to promote the secretion of hyaluronic acid (>0.3 ng/mL) at a concentration of 8%-20%.

### Example 6

Evaluation of the Immunomodulatory Activity of Dendrobium officinale Oligosaccharides

1. Experimental Cells and Reagents:

RAW264.7 cells, TNF-α kit, IL-6 kit, distilled water.

2. Experimental methods:

The logarithmic-growth RAW264.7 cells were taken and inoculated into 12-well plates after cell counting. The plate was placed in an incubator overnight, and then the cell supernatant was discarded. Different concentrations of Dendrobium officinale oligosaccharides, lipopolysaccharides and basal medium 1640 were added under aseptic operation, and the supernatant after 24 h of intervention was collected and stored at −20° C. for later use. The changes in TNF-α and IL-6 secretion were detected according to the instructions of the ELISA kit. The OD value was measured by a microplate reader with a detection wavelength of 450 nm. The OD value was substituted into the regression equation of the standard curve to calculate the increase rate of TNF-α and IL-6 secretion.

3. Experimental Results:

As shown in Table 7.

TABLE 7

| | Final reaction | | Final reaction | |
|---|---|---|---|---|
| | concentration | TNF-α | concentration | IL-6 |
| Sample | (μg/mL) | pg/mL | (μg/mL) | pg/mL |
| Blank control | | 64.71 | | 1013.72 |
| LPS | | 1085.13 | | 1406.19 |
| Dendrobium | 12.5 | 53.86 | 200 | 1117.33 |
| officinale | | | | |
| oligosaccharide | | | | |
| Dendrobium | 6.25 | 35.66 | 100 | 1376.42 |
| officinale | | | | |
| oligosaccharide | | | | |
| Dendrobium | 3.125 | 1062.29 | 50 | 1593.01 |
| officinale | | | | |
| oligosaccharide | | | | |

4. Conclusion:

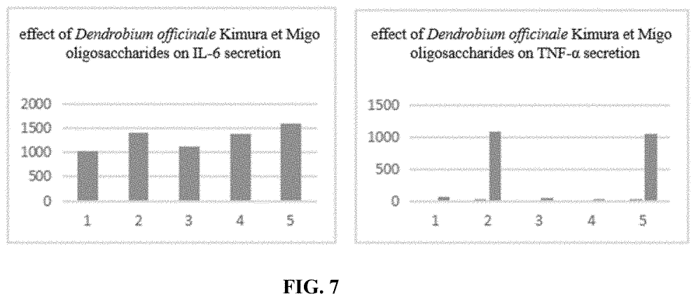

According to the data in Table 7, FIG. 7 is made for a clearer comparison. As can be seen from FIG. 7 and Table 7, the effects of Dendrobium officinale oligosaccharide on the immunomodulatory activity of RAW264.7 cells is evaluated. The results show that the secretion of TNF-α by RAW264.7 macrophages is significantly increased at a concentration of 3.125 μg/mL, which is equivalent to the positive control LPS. The secretion of IL-6 by RAW264.7 macrophages can be significantly increased at a concentration of 50 μg/mL, which is equivalent to the positive control LPS.

### Example 7: Glycosylation of Fatty Acids

1. Experimental Reagents:

Dendrobium officinale oligosaccharides, fatty acids, NaBH₄, MeOH, metal salts, AlCl₃, THF, catalysts, etc.

2. Experimental Methods and Results:

1) Reducing a C₆ fatty acid into a fatty alcohol: 10 mmol of fatty acid, 20 mmol of NaBH₄, 40 mmol of AlCl₃, 27 mL of THF and 3 mL of MeOH were reacted at a temperature of 100° C. for a time of 8 h with a yield of 90%;

2) Glycosylation: the reduced fatty alcohol and the Dendrobium officinale oligosaccharide were subjected to a glycosylation reaction with a molar ratio of the alcohol and the saccharide of 1:(8-10); the catalyst was cetyl ammonium bromide; the reaction was conducted under a temperature of 110-115° C. for a time of 3 h with a yield of 80%.

3. Results Discussion:

The reaction results show that Dendrobium officinale oligosaccharides with terminal glycosides can also undergo the glycosylation reaction with fatty acids.

### Example 8

This example provides an anti-aging or immunomodulatory face cream, comprising components in mass percentage: 0.4-0.6% of Dendrobium officinale stock solution rich in oligosaccharides, 7.5-8.5% of stearic acid, 1.5-2.5% of C₁₆ alcohol, 1.5-2.5% of self-emulsifying monoglyceride, 1.5-2.5% of hydrogenated lanolin, 11-13% of liquid paraffin, 6-8% of glycerin, 1-2% of an emulsifier, 0.1-0.3% of a preservative, 0.1-0.3% of an essence, and the balance of water. The face cream of the above formula was prepared according to a conventional method for preparing cosmetics.

### Example 9

This example provides an anti-aging or immunomodulatory emulsion, comprising components in mass percentage: 0.4-0.6% of Dendrobium officinale stock solution rich in oligosaccharides, 1.3-1.5% of stearic acid, 0.08-0.12% of cetyl alcohol, 1.7-1.9% of 2-ethyl alcohol cetyl stearate, 0.1-0.3% of isopropyl myristate, 0.9-1.1% of 2-hexyl-1-decanol, 7-8% of liquid paraffin, 2.5-3.5% of glycerol, 7-9% of propylene glycol, 0.08-0.12% of triethanolamine, 0.3-0.4% of a carboxyvinyl polymer, 1.5-2.5% of Arlacel 165, 0.1-0.3% of a preservative, 0.1-0.3% of an essence, and the balance of water; The emulsion of the above formula was prepared according to a conventional method for preparing cosmetics.

### Example 10

This example provides an anti-aging or immunomodulatory toner, comprising components in mass percentage: 1-10% of Dendrobium officinale stock solution rich in oligosaccharides, 0.3-0.4% of sodium polyacrylate, 3.5-4.5% of glycerol, 2-3% of 1,3-butanediol, 0.4-0.6% of vitamin B₅, 0.4-0.6% of arbutin, 0.04-0.06% of EDTA-Na₂, and the balance of deionized water; the method for preparing the toning lotion is as follows: the sodium polyacrylate was dissolved in water, stirred to fully swelling, then the remaining ingredients were added, and continued to stirring; then the Dendrobium officinale stock solution rich in oligosaccharides was added thereto, the mixture was stirred, and then deionized water was added to a constant volume to obtain the toner.

A preferred solution is as follows: 0.35 g of sodium polyacrylate was dissolved in 70 mL of water, stirred to fully swelling, then the remaining ingredients were added, and continued to stirring; then 10% of the Dendrobium officinale stock solution rich in oligosaccharides obtained in Example 1 was added thereto, the mixture was stirred uniformly, and then deionized water was added to 100 mL and packed.

### Example 11

This example provides an anti-aging or immunomodulatory repair cream, comprising components in mass percentage: 5-7% of glycerin, 1.4-1.6% of carbomer, 1.4-1.6% of triethanolamine, 5.5-6.5% of propylene glycol, 0.15-0.25% of ethyl p-hydrobenzoate, 9-11% of Dendrobium officinale stock solution rich in oligosaccharides, 0.4-0.6% of an essential oil, and the balance of deionized water.

The method for preparing the repair cream is as follows: the carbomer was taken in an appropriate amount of deionized water, the mixture was stirred uniformly, let stand overnight to fully swelling, then glycerin was added, and the pH value was adjusted with triethanolamine to increase the viscosity of the gel matrix to obtain a carbomer gel; the Dendrobium officinale stock solution rich in oligosaccharides, propylene glycol and deionized water were mixed evenly, then the carbomer gel, essential oil and ethyl p-hydrobenzoate were added thereto, the resulting mixture was stirred until uniform and delicate, distilled water was added to a sufficient amount, and the mixture was grinded evenly to obtain a repair cream.

A preferred solution is as follows: 6 g of glycerin, 1.5 g of carbomer, 1.5 g of triethanolamine, 6 g of propylene glycol, 0.2 g of ethyl p-hydrobenzoate, 10% of Dendrobium officinale stock solution rich in oligosaccharide obtained in Example 1, and an appropriate amount of deionized water were added to a total weight of 100 g. The preparation method is as follows: the carbomer was taken in an appropriate amount of deionized water, the mixture was stirred uniformly, let stand overnight to fully swelling, then glycerin was added, and the pH value was adjusted with triethanolamine to increase the viscosity of the gel matrix to obtain a carbomer gel; a prescription amount of tanshinone extract, propylene glycol and deionized water were mixed evenly, then the carbomer gel, 0.5% of an essential oil and ethyl p-hydrobenzoate were added thereto, the resulting mixture was stirred until uniform and delicate, distilled water was added to a sufficient amount, and the mixture was grinded evenly to obtain a repair cream.

The above are only preferred embodiments of the present disclosure, and are not intended to be used to limit the present disclosure in any form. Any simple amendments, equivalent changes and modifications made to the above embodiments based on the technical essence of the present disclosure still fall within the scope of the technical solutions of the present disclosure.

1.-16. (canceled)

17. A Dendrobium officinale oligosaccharide, comprising 3-9 glycoside residues and a glucose residue at a non-reducing end.

18. The Dendrobium officinale oligosaccharide according to claim 17, wherein the Dendrobium officinale oligosaccharide comprises 6 glycoside residues.

19. The Dendrobium officinale oligosaccharide according to claim 17, wherein the Dendrobium officinale oligosaccharide has a structure represented by formula (I):

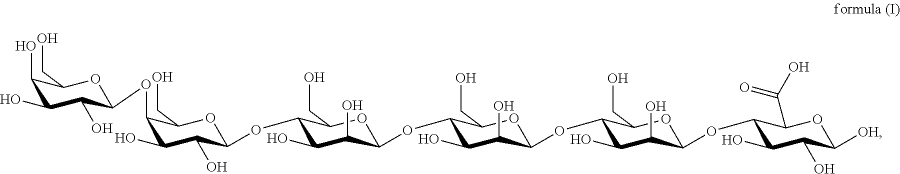

20. The Dendrobium officinale oligosaccharide according to claim 17, wherein the glycoside residue comprises a galactose residue, a mannose residue and a glucose residue.

21. The Dendrobium officinale oligosaccharide according to claim 20, wherein a molar ratio of the galactose residue, the mannose residue, and the glucose residue is (3-2):(2-1):(2-1).

22. The Dendrobium officinale oligosaccharide according to claim 21, wherein a molar ratio of the galactose residue, the mannose residue and the glucose residue is 2:1:1.

23. A Dendrobium officinale oligosaccharide derivative, wherein the Dendrobium officinale oligosaccharide derivative is generated by reacting the glucose residue of the Dendrobium officinale oligosaccharide according to claim 16 with a hydrophobic residue, and the hydrophobic residue comprises any one of linear or branched C₁-C₁₈ alkyl, C₁-C₁₈ alkyl carboxylic acid, C₁-C₁₈ alkyl amine, C₁-C₁₈ aryl alkyl and C₁-C₁₈ alkyl amide, or the hydrophobic residue comprises any one of benzoyl, phenylacetyl and phenylpropionyl.

24. A method for preparing anti-aging or immunomodulatory products, comprising steps of using the Dendrobium officinale oligosaccharide according to claim 17.

25. The method according to claim 24, wherein the products comprises medicines, foods, formulations and cosmetics.

26. The method according to claim 25, wherein a type of the cosmetics comprises a face cream, an emulsion, a toning lotion, or a repair cream.

27. A method for extracting the Dendrobium officinale oligosaccharide according to claim 17, comprising:
1) preparation of a Dendrobium officinale powder: drying a stem of Dendrobium officinale, then smashing and sieving to obtain a Dendrobium officinale powder;
2) preparation of a water extract of Dendrobium officinale: mixing the Dendrobium officinale powder with water, subjecting to a boiling water bath for one or more times, and then merging water extracts to obtain a water extract of Dendrobium officinale;
3) preparation of a concentrated water extract of Dendrobium officinale: centrifuging the water extract of Dendrobium officinale, and then concentrating under reduced pressure to obtain a concentrated water extract of Dendrobium officinale;
4) preparation of a redissolving liquid: adding ethanol to the concentrated water extract of Dendrobium officinale, precipitating, centrifuging and then discarding a supernatant to obtain a precipitate; adding water to the obtained precipitate for redissolving to obtain a redissolving liquid;
5) preparation of an aqueous solution of Dendrobium officinale oligosaccharide: deproteinizing the redissolving liquid, and freeze-drying to obtain a crude polysaccharide of Dendrobium officinale; adding water to dissolve the crude polysaccharide of Dendrobium officinale, then adding cellulase for a reaction, followed by inactivating and centrifuging, and discarding a precipitate to obtain an aqueous solution of Dendrobium officinale oligosaccharide;
6) preparation of the Dendrobium officinale oligosaccharide: purifying the aqueous solution of Dendrobium officinale oligosaccharide to obtain the Dendrobium officinale oligosaccharide.

28. The method according to claim 27, wherein,
in step 1), a temperature for the drying is 50-60° C.; a moisture mass content of the stem of Dendrobium officinale after the drying is less than 6%; a mesh number of the sieving is 20-40 meshes;
in step 2), the Dendrobium officinale powder and water are mixed in a mass-volume ratio of 1:(50-80) g/mL; the boiling water bath is performed for 2-3 times; a time for each boiling water bath is 1-2 h;
in step 3), the centrifugation is conducted under conditions of 3000-4000 rpm/min, 8-12 min and room temperature; the concentration under reduced pressure is conducted under conditions of a rotary evaporation method, an ethanol recycling temperature of less than 50° C. and a pressure of 0.7 Pa-0.9 Pa; a material-liquid mass-volume ratio of the concentrated water extract of Dendrobium officinale is 1:(5-10) g/mL.

29. The method according to claim 27, wherein,
in step 4), the addition of ethanol to the concentrated water extract of Dendrobium officinale and the precipitation are specifically conducted as follows: adding ethanol with a volume concentration of 95% to the concentrated water extract of Dendrobium officinale until an alcohol concentration of a mixture is 70%-80% in volume percentage, and then precipitating overnight;
the centrifugation is conducted under conditions of 3500-4500 rpm, 18-22 min and room temperature; a weight-volume ratio of the precipitate and water for the redissolving is 1:(4-6) g/mL;
in step 5), the deproteinization of the redissolving liquid is conducted by a Sevage method;
a temperature for the freeze-drying is 45° C.;
the addition of water to dissolve the crude polysaccharide of Dendrobium officinale is conducted as follows: adding water to the crude polysaccharide of Dendrobium officinale for dissolving until 4.5-5.5 mg/mL;
an added amount of the cellulase is 2%-4% of a weight of the crude polysaccharide of Dendrobium officinale;
after adding the cellulase, the reaction is conducted under a reaction temperature of 55-65° C. for a reaction time of 2 h-4 h;
the inactivation is conducted by boiling for 25-35 min;
the centrifugation is conducted under conditions of 3500-4500 rpm, 15-25 min and room temperature;
an oligosaccharide content in the aqueous solution of Dendrobium officinale oligosaccharide is greater than 80%;
in step 6), the purification is conducted by passing through a G75 gel column; a purity of the Dendrobium officinale oligosaccharide is 99.86%.

30. A method for preparing the Dendrobium officinale oligosaccharide derivative according to claim 23, comprising:
1) reducing a fatty acid into a fatty alcohol;
2) glycosylating the fatty alcohol with the Dendrobium officinale oligosaccharide to obtain the Dendrobium officinale oligosaccharide derivative.

31. The method according to claim 30, wherein in step 1), a reaction process of the reduction of a fatty acid into a fatty alcohol is as follows: mixing 9-11 mmol of the fatty acid, 18-22 mmol of NaBH₄, 38-42 mmol of AlCl₃, 25-29 ml of THF and 2-4 ml of MeOH for a reaction, and the reaction is conducted under a temperature of 95-105° C. for a time of 7.5-8.5 h with a yield of 88-92%;
in step 2), a reaction process of the glycosylation is as follows: mixing the alcohol and the saccharide in a molar ratio of 1:(8-10), and then using cetyl ammonium bromide as a catalyst for catalyzation, and the reaction is conducted under a temperature of 110-115° C. for a time of 2.5-3.5 h with a yield of 78-82%.

32. An anti-aging or immunomodulatory product, wherein the anti-aging or immunomodulatory product is a face cream, an emulsion, a toning lotion or a repair cream;
the face cream comprises in mass percentage: 0.4-0.6% of a Dendrobium officinale stock solution rich in oligosaccharides, 7.5-8.5% of stearic acid, 1.5-2.5% of C₁₆ alcohol, 1.5-2.5% of self-emulsifying monoglyceride, 1.5-2.5% of hydrogenated lanolin, 11-13% of liquid paraffin, 6-8% of glycerin, 1-2% of an emulsifier, 0.1-0.3% of a preservative, 0.1-0.3% of an essence, and the balance of water;
the emulsion comprises in mass percentage: 0.4-0.6% of a Dendrobium officinale stock solution rich in oligosaccharides, 1.3-1.5% of stearic acid, 0.08-0.12% of cetyl alcohol, 1.7-1.9% of 2-ethyl alcohol cetyl stearate, 0.1-0.3% of isopropyl myristate, 0.9-1.1% of 2-hexyl-1-decanol, 7-8% of liquid paraffin, 2.5-3.5% of glycerol, 7-9% of propylene glycol, 0.08-0.12% of triethanolamine, 0.3-0.4% of a carboxyvinyl polymer, 1.5-2.5% of Arlacel 165, 0.1-0.3% of a preservative, 0.1-0.3% of an essence, and the balance of water;
the toning lotion comprises in mass percentage: 1-10% of a Dendrobium officinale stock solution rich in oligosaccharides, 0.3-0.4% of sodium polyacrylate, 3.5-4.5% of glycerol, 2-3% of 1,3-butanediol, 0.4-0.6% of vitamin B₅, 0.4-0.6% of arbutin, 0.04-0.06% of EDTA-Na₂, and the balance of deionized water;
the repair cream comprises in mass percentage: 5-7% of glycerin, 1.4-1.6% of carbomer, 1.4-1.6% of triethanolamine, 5.5-6.5% of propylene glycol, 0.15-0.25% of ethyl p-hydrobenzoate, 9-11% of a Dendrobium officinale stock solution rich in oligosaccharides, 0.4-0.6% of an essential oil, and the balance of deionized water.

33. The Dendrobium officinale oligosaccharide according to claim 19, wherein the Dendrobium officinale oligosaccharide comprises 6 glycoside residues.

34. A method for preparing anti-aging or immunomodulatory products, comprising steps of using the Dendrobium officinale oligosaccharide derivative according to claim 23.

35. The method according to claim 34, wherein the products comprises medicines, foods, formulations and cosmetics.

36. The method according to claim 35, wherein a type of the cosmetics comprises a face cream, an emulsion, a toning lotion or a repair cream.
